(19) United States
(12) Patent Application Publication
(10) Pub. No.: US 2005/0112649 A1
(43) Pub. Date: May 26, 2005

(54) Sensitive and self-calibrating multi-test system the detection and identification of enterohemorrhagic escherichia coli (EHEC)

(75) Inventors: Nabil Chbouki, Arlington, VA (US); Noreddine Benkerroum, Rabat (MA)

Correspondence Address: Nabil Chbouki, 4500 S. Four Mile Run Drive, #611, Arlington, VA, 22204, US

(73) Assignee: Nabil Chbouki, Arlington, VA (US)

(21) Appl. No.: 10/960,333

(22) Filed: Oct. 7, 2004

Related U.S. Application Data
(60) Provisional application No. 60/509,436, filed on Oct. 8, 2003.

(51) Int. Cl. C12Q 1/68; G01N 33/554; G01N 33/569

(52) U.S. Cl. 435/6; 435/7.32

(57) ABSTRACT

We propose a novel high sensitivity multi-test detection and identification system for EHEC. The integrated testing system uses, in a single run, multiple molecular signatures and receptor families to characterize the genetic, immunological, and toxin fingerprints of the pathogen. The multiplicity of signatures enhances the sensitivity of the test and provides intrinsic control to eliminate false positives. The test can be adapted, through the appropriate selection of antibodies, receptors and probes for other pathogens and clinical conditions.

### CROSS REFERENCES TO RELATED APPLICATIONS

This application claims the benefit of U.S. Provisional application Ser. No. 60/509,436, filed on Oct. 8, 2003.

### CITED REFERENCES

1. Jones, U. L. 1999. Potential health risks associated with the persistence of Escherichia coli O0157 in agricultural environments. Soil Use and Management. 15:76-83.
2. Armstrong, G. L., J. Hollingsworth, and J. G. Morris. 1996. Emerging foodborne pathogens: Escherichia coli O157:H7 as a model of entry of a new pathogen into the food supply of the developed world. Epidemiol. Rev. 18:29-51.
3. Duffy, G. 2003. Verotoxinogenic Escherichia coli in animal faeces, manures and slurries. J Appl. Microbiol. 94:94S-103S
4. Chinen, I., J. D. Tanaro, E. Miliwebsky, L. H. Lound, G. Chillemi, S. Ledri, A. Baschkier, M. Scarpin, E. Manfredi, and M. Rivas. 2001. Isolation and characterization of Escherichia coli O157:H7 from retail meats in Argentina. J. Food Prt. 64:1346-1351.

### BACKGROUND OF THE INVENTION

1. Field of the Invention

Rapid diagnostic testing for food safety, veterinary and biomedical applications using multiple molecular signatures.

2. Background Art

Enterohemorrhagic Escherichia coli (EHEC); also called Shiga-toxin-producing E. coli (STEC), is one of the pathotypes of the species known to cause serious food borne diseases because of the widespread of the pathogen in foods and agricultural environments, its low infective dose (i.e., 10 to 100 CPUs)⁽¹⁾ and its ability to express different virulence factors including shiga-like toxins (Stx1 and/or Stx2), intimin and enterohemolysins, thereby causing a wide range of diseases varying from uncomplicated diarrhea to severe complications including thrombocytopenic purpura (TCP), hemorrhagic colitis (HC) and hemolytic uremic syndrome (HUS).

Ruminants, particularly cattle and sheep, have been identified as major natural reservoirs for EHEC strains, and foods of bovine origin were the most common sources of STEC transmission to human. Nonetheless, other foods including drinking water, fruits, vegetables, cider and juices as well as man-to-man or animal-to-man contacts have been implicated in the transmission of the disease^{(2,3)}. E. coli strains of different “O” serotypes have been reported to produce shiga toxins or to be involved in human diseases; however, the serotype O157:H7 remains the prototype of EHEC and accounts for 70 to 80% of recognized clinical cases of the disease. Nonetheless, the incidence of non-O157 EHEC in foods seems to be overlooked due to the lack of appropriate techniques to detect EHEC strains other than those of the 0157 serotype.

Immunological tests have been extensively used to detect E. coli O157 and have yielded fairly satisfactory results though having several limitations. Agglutination with anti-O157 antiserum or with a commercial latex agglutination test kit for presumptive identification of E. coli O157 was the most frequently used. The latter was even recommended, in conjunction with other conventional methods, by official food safety organisms such as Food and Drug Administration (USA), Canadian Food Inspection Agency (Canada) and by ISO. The main disadvantages of immunoassays seem to be the time that it takes to obtain pure cultures that can be tested and yet; the results are not conclusive as immunoassays may yield false positive results due to cross-reaction with other bacteria including non-EHEC E. coli strains and members of group N Salmonellae (e.g., Salmonella urbatia) with the polyclonal antiserum raised against E. coli O157. Therefore, further confirmation of the strain identity by morphological and biochemical tests as well as characterization of the virulence factors and/or their genes is usually required.

Genetic techniques including DNA hybridization, whole cell blot or PCR have been developed for detection of EHEC. These assays utilize primers and probes that hybridize specifically to complementary sequences found in the STEC genome. Among those, PCR has proven the most reliable, sensitive and specific. It is used to characterize shiga-toxin genes regardless of the strain serotype and, hence, can detect all verotoxigenic E. coli (STEC) types, including E. coli O157:H7. It is also used to characterize the genes encoding for intimin and enterohemolysins in presumptive purified EHEC strains. While these assays are specific and sensitive, they are expensive, tedious and require technical skills to be carried out properly.

Besides the above-mentioned tests, in vitro tests are used to provide evidence of the expression of the main virulence factors in putative isolated EHEC strains (i.e., toxin and enterohemolysins). Detection of the toxins is carried out by the Vero cell assay⁽⁴⁾. Immunological tests (i.e., immunoagglutination test kit or Enzyme-liked-immunosorbent-assay; ELISA) are also used to provide such evidence. On the other hand, the production of enterohemolysins is evidenced by the lysis of washed sheep red blood cells by in vitro testing. Though these tests are reliable when positive results are obtained, their interpretation is uncertain when they yield negative results since the strain may carry the corresponding genes without being able to express them under certain conditions. Therefore, further confirmation by genetic characterization is usually needed and results in additional labor, cost and time.

Obviously, there is an urgent need to develop new rapid, sensitive, simple and reliable techniques that can detect E. coli O157:H7 and/or other EHEC serotypes so that the risk of E. coli O157:H7 outbreaks can be properly evaluated and reduced.

### SUMMARY OF THE INVENTION

We propose a novel high sensitivity multi-test detection and identification system for EHEC. The integrated testing system uses, in a single run, multiple molecular signatures and receptor families to characterize the genetic, immunological, and toxin fingerprints of the pathogen. The multiplicity of signatures enhances the sensitivity of the test and provides intrinsic control to eliminate false positives.

The test offers fast time-to-results and provides the practitioner with clear diagnostic for on-the-spot decision-making and action. The test also uses a simplified sample preparation method and assay protocol to reduce sample volume and preparation time, reagent consumption, and cost, which are critical for successful field testing.

The proposed integrated testing system is intended as a diagnostic tool for the food industry, veterinary testing and biomedical applications.

It can be used in conjunction with pulsed-field gel electrophoresis to provide a convenient tool for epidemiological studies as well.

### DETAILED DESCRIPTION OF THE INVENTION

We propose to run simultaneously 3 or more tests now being used separately to confirm the identity, the serotype and the presence of virulence factors. The tests to be included in the battery are the following:
Presence of E. coli O157 using anti-O157 antibody as a capture
Production of Stx1 or Stx2 toxins using the receptor Gb3 or anti-Stx antibody as a capture
The presence of stx1, stx2, eaeA, and/or ehly genes using the corresponding available primers as captures.

Receptors and probes are immobilized on individually addressable microelectrodes or micro-spots 10 to 700 μm in diameter on a generic biochip capable of several dozens to several hundred tests. The biochip can be a micro-printed glass slide or a planar device with lithographically imprinted features. Planar-based micro-spots can take on a variety of configurations like a simple longitudinal strip, a coiled spot with high active surface area, or a micro-disk resonator. Detection schemes can use fluorescence or rely on direct and label-free optical-based detectors using evanescent field sensing, interferometers or microbalances.

Binding signals will be transformed into electric ones, amplified and processed. Built-in intelligence will analyze the results and can use a look-up table to eliminate false positives and yield a clear and actionable diagnosis.

This technique will be used to test for the presence of EHEC strains and/or their virulence factors directly after a seven-hour enrichment or on pure cultures of suspected colonies. In both cases, the procedure saves time and improves the accuracy of the obtained results at reduced cost as compared to the conventional methods.

The proposed technology takes advantage of the well-established sensitivity and specificity of DNA-based methods (e.g., PCR) and the availability of the corresponding primers to detect the genes encoding for the virulence factors (i.e., stx, eaeA. and ehly) without need to amplify the genes and to perform the gel electrophoresis as is done in the PCR thus simplifying the procedure, and reducing cost and labor.

The technique will use the specific binding of shiga toxins to the Gb3 receptor as means to detect the toxins. These toxins have been detected either directly by in vitro Vero cell assay, by an immunoagglutination test kit using sensitized latex or by ELISA or indirectly by PCR techniques to detect the corresponding genes (stx1 or stx2). All these alternative tests carried out on pure cultures and hence can only be preformed after different steps including pre-enrichment, enrichment, isolation and purification. In the proposed technique, polymixin B will be added to stimulate toxin production by putative EHEC during the enrichment step to allow direct detection of the toxin(s) if any in enrichment broth.

The technique will discriminate between the occurrences of O157 and non-O157 EHEC strains in foods.

The method of combining multiple molecular signatures can be applied to other pathogens and clinical conditions by using the same generic platform of chips and micro-spots immobilized with the appropriate antibodies, receptors and DNA/RNA probes.

### PREFERRED EMBODIMENT

The proposed testing system will integrate at least six tests per pathogen with 2 or 3 replicates on the same chip requiring at least 18 micro-spots in addition to one or two reference micro-spots. A one-cm² chip can provide the capacity to run several samples in a single run. Micro-spots will provide dense features with small diameters of 10 μm for confined reaction volumes and enhanced sensitivity. A micro-spot will preferably provide a 3-D configuration to increase the active surface and the intensity of the binding signal through the additional spatial confinement.

Receptors are immobilized in a recognizable configuration so that results from each micro-spot can be appropriately analyzed. Microfluidic and kinetic conditions can lead to a special spatial configuration to enhance sensitivity and speed.

A label-free integrated optical biosensing scheme using evanescent field sensing can be used. This detection scheme leverages the capability of planar waveguides, provides high surface sensitivity, and can be implemented in many low cost configurations.

Other simple and generic platforms, like MEMS based cantilevers and piezoelectric microbalances, can be used as well.

1. A multiple molecular signatures test comprised of a combination of the genetic, immunological and toxin fingerprints of a pathogen or clinical condition for accurate detection and identification.

2. The test as recited in claim 1 wherein the molecular signatures use a combination of antibodies, peptides, nucleic acids, or any other organic and inorganic receptors.

3. An EHEC test with a battery of at least 3 tests using anti-O157 antibodies, Gb3 or anti-Stx toxin antibodies, and probes for stx1, stx2, eaeA, and/or ehly genes.
